(19) United States
(12) Patent Application Publication
(10) Pub. No.: US 2019/0036619 A1
(43) Pub. Date: Jan. 31, 2019

(54) MEDICAL DEVICE WITH A COMMUNICATIONS INTERFACE CONFIGURED FOR PROTECTION OF PATIENTS AND OPERATORS

(71) Applicant: Fresenius Medical Care Deutschland GmbH, Bad Homburg (DE)

(72) Inventors: Stefan Saal, Schonungen (DE); Frank Hedmann, Volkach (DE); Martin Buda, Dittelbrunn (DE); Torsten Hochrein, Eschenau (DE)

(21) Appl. No.: 16/036,496

(22) Filed: Jul. 16, 2018

(30) Foreign Application Priority Data
Jul. 27, 2017 (DE) 10 2017 007 033.4

(51) Int. Cl. H04B 10/80; A61M 1/28

(52) U.S. Cl. CPC H04B 10/807; A61M 2205/3576; A61M 1/28

(57) ABSTRACT

A medical device, such as a hemodialysis or peritoneal dialysis machine, has at least one housing, electrical and/or electronic components arranged inside the housing, and a user interface. The medical device has a first receiving device accessible outside of the housing and a first communications interface for receiving a proximal end of a medium carrying communication signals. A distal end of the medium is provided for connection to a second communications interface associated with a communications network. Components of the medical device are communicatively connected to the first communications interface. A communication link between the distal end of the medium and the components of the medical device has at least two segments. At least one of the segments transmits communication signals in an electrically non-conductive manner and has electrical isolation between the input and output and/or between the output and input of the segment with a predetermined first dielectric strength.

### CROSS-REFERENCE TO RELATED APPLICATIONS

Priority is claimed to German Patent Application No. DE 10 2017 007 033.4, filed on Jul. 27, 2017, the entire disclosure of which is hereby incorporated by reference herein.

### FIELD

The present invention relates to a medical device with a communications interface, which is configured for protection of patients and operators from hazardous electrical voltages and currents and for a low sensitivity to interference.

### BACKGROUND

Medical devices are increasingly equipped with interfaces for communication over a network communications infrastructure, for example, for transmitting equipment data and patient data detected during operation to databanks or servers for storage and/or processing or to also enable remote maintenance functions, such as updating a control software or operating parameters, for example.

Medical devices include, for example, dialysis machines (e.g., hemodialysis or peritoneal dialysis machines), machines for monitoring cardiovascular parameters in patients, infusion equipment and the like.

Dialysis machines are machines for extracorporeal treatment of blood, in which blood from a patient is sent through a first fluid line of a blood treatment component, treated by the blood treatment component and then returned to the patient through a second fluid line. Examples of such blood treatment machines include hemodialysis machines in particular. One such blood treatment machine is the subject matter of U.S. Pat. No. 6,676,621, the contents of which are incorporated by reference herein.

Dialysis is a process for purifying the blood of patients in chronic or acute renal failure. Dialysis includes methods which have an extracorporeal blood circulation, such as hemodialysis, hemofiltration or hemodiafiltration, as well as methods which does not have an extracorporeal blood circulation, such as peritoneal dialysis.

In extracorporeal blood circulation in hemodialysis, blood is passed through the blood chamber of a dialysis machine, which is separated from a dialysis fluid chamber by a semipermeable membrane. Dialysis fluid containing blood electrolytes in a certain concentration flows through the dialysis fluid chamber. The substance concentration of blood electrolytes in the dialysis fluid corresponds to the concentration of blood electrolytes in a healthy person's blood. During the treatment, the patient's blood and the dialysis fluid are circulated on respective sides of the semipermeable membrane, usually in countercurrent with one another, at a predefined flow rate. Urinary excretion substances diffuse through the membrane of the blood chamber into the chamber for dialysis fluid, while electrolytes present in the blood and in the dialysis fluid diffuse from the higher-concentration chamber to the lower-concentration chamber. When a pressure gradient is applied by a pump, for example, from the blood side to the dialysate side of the dialysis membrane, water passes from the patient's blood through the dialysis membrane and into the dialysate circulation, thereby withdrawing dialysate from the dialysis circulation downstream from the dialysis filter on the dialysate side. This process, also known as ultrafiltration, leads to the desired removal of water from the patient's blood.

In hemofiltration, ultrafiltrate is removed from the patient's blood by applying a transmembrane pressure in the dialysis machine without passing the dialysis fluid over the side of the membrane of the dialysis machine opposite the patient's blood. In addition, a sterile and pyrogen-free substituate solution can be added to the patient's blood. This is called predilution or postdilution, depending on whether the substituate solution is added upstream or downstream from the dialysis machine. The mass exchange in hemofiltration takes place by convection.

Hemodiafiltration combines hemodialysis and hemofiltration methods. There is diffusive mass exchange between the patient's blood and the dialysis fluid across the semipermeable membrane of a dialysis machine, and plasma water present in the blood is filtered via a pressure gradient on the membrane of the dialysis machine.

Hemodialysis, hemofiltration and hemodiafiltration methods are usually carried out with automatic hemodialysis machines.

Withdrawal of blood from the patient and introduction into the extracorporeal blood circulation take place via blood pumps, for example, peristaltic pumps or impeller pumps, which pump blood from the patient's bloodstream to the dialysis machine. The purified blood is returned by a similar pathway via corresponding blood pumps. In the extracorporeal blood circulation, blood is passed through tubing lines, which can also be connected to other devices, for example, sensors for detecting properties of the blood, the blood pressure, etc., in addition to being connected to the dialysis machine.

Dialysis fluid is conveyed out of a storage container or a central line via one or more dialysate pumps, sent to an inlet to the dialysis machine and removed from the dialysis machine at a corresponding outlet for disposal. The dialysate pumps may also be peristaltic pumps or impeller pumps.

Plasmapheresis is a blood treatment process, in which a patient's blood is separated into blood plasma and its corpuscular constituents (cells). The separated blood plasma is purified or replaced by a replacement solution, and the purified blood plasma or replacement solution is returned to the patient.

In peritoneal dialysis, a patient's abdominal cavity is filled with a dialysis fluid through a catheter passed through the abdominal wall. This dialysis fluid has a concentration gradient of blood substances such as electrolytes (for example, sodium, calcium and magnesium) with respect to endogenous fluids. Toxins present in the body pass from the peritoneal blood vessels and into the abdominal cavity by passing through the peritoneum, which acts as the membrane.

After a few hours, the dialysis fluid in the patient's abdominal cavity, now containing the toxins transferred from the patient's body, is replaced. Water can be transferred into the dialysis fluid from the patient's blood by passing through the peritoneum due to osmotic processes, thereby removing the water from the patient.

The peritoneal dialysis process is usually carried out with the help of automatic peritoneal dialysis machines.

Dialysis machines, as an example of complex medical machines, have extensive functions and a plurality of electrical and/or electronic components. Medical devices such as dialysis machines are equipped with at least one control unit or one control device for controlling these functions as well as the electrical and/or electronic components. These control devices may include one or more CPUs (central processing units) or microcontrollers, which are controlled by software programs. It will be appreciated that methods described herein may be carried out by one or more control units. A plurality of control units carrying out methods described herein individually or in association may be considered to be a single control unit or multiple control units. The software programs are usually stored in an internal memory device. A plurality of memory devices may be utilized for storing other information, such as treatment data. The control unit controls one or more pumps during a treatment in order to trigger a flow of blood through the extracorporeal blood circulation and the blood treatment device. Furthermore, the control unit controls one or more sensors, for example, pressure sensors and flow sensors, to detect the course of the treatment and regulate it, if necessary, and also to detect critical states in the patient or machine and to adjust or terminate or interrupt the treatment accordingly.

During a treatment, which typically lasts between four and five hours, a plurality of machine-related data and patient-related data is generated and is to be stored for the purposes of documentation and analysis. Such data can be saved on a data medium connected permanently or temporarily to the dialysis machine, but the data should also be transferred to a database at a subsequent point in time in order to permit further analysis in comparison with other data. Furthermore, due to the limited memory capacity of data media permanently or temporarily connected to the dialysis machine, the data media should be cleared regularly in order to be able to record new data.

Continuous or packet-wise transmission of data to the database during a treatment minimizes or eliminates some of the working steps, such as manual transfer of treatment data between the dialysis machine and a clinic or hospital using data storage media (such as, e.g., universal serial bus (USB) memory sticks or patient cards), thus reducing possible error sources. Network interfaces provided in modern medical devices allow for connection to a corresponding network communications infrastructure of a medical facility.

Modern medical facilities usually have a complex and heterogeneous network communications infrastructure connecting a plurality of different machines to one another and/or to central servers and databases. “Heterogeneous” here means that, first of all, there is a network communication among medical machines with one another and with dedicated databases and servers, for example, for saving and processing medical data and, second, there is a network communication among general information systems and machines. The network communications infrastructure comprises a plurality of network nodes that direct and deflect communications, e.g., routers and switches connected to connecting lines.

The network communications infrastructure of a medical facility and a medical machine associated therewith should be in compliance with the statutory standards for electrical safety, for example as defined in European Standard EN 60601-1, among others. Comparable standards also exist in countries outside of the scope of European standards.

The EN 60601-1 standard defines, among other things, general requirements for the basic safety of electrical devices or systems, which are connected to a power supply network and are intended for diagnosis, treatment or monitoring of patients and are in direct physical or electrical contact with the patient. Depending on the type of medical device, other requirements may also be defined in other supplementary standards, under some circumstances in such a way as to cancel, amend or supplement the EN 60601-1 requirements.

Special attention is devoted to patient safety and the safety of the medical personnel as users and operators of the electrical machines. The safety of electrical machines is determined in particular by insulation, air gaps and creep gaps, the properties of components and grounding. In some places in the description, the insulation, air gaps and creep gaps are also referred to as electrical or galvanic separation. The standard requires that medical equipment coming in contact with patients must be equipped with two independent safety measures (MOP, means of protection), so that electric safety is still ensured even in the event of failure of one of the measures. These insulation stages are defined as insulation requirements for devices to protect the user/operator (means of operator protection, MOOP) or the patient (means of patient protection, MOPP). Based on the increased risks when patients come in contact with electrical or electronic equipment, higher demands are made of MOPP for protection of patients than of MOOP for protection of the user/operator.

EN 60601-1 defines MOOP and MOPP specifications for the dielectric strength of insulation and the length of the creep zone between voltage-carrying portions on both sides of the insulation. A 1 H MOOP classification requires a 1500 VAC dielectric strength of the insulation and a 2.5 mm creep zone. The requirements are doubled for a 2 H MOOP classification, so this yields a 3000 VAC dielectric strength of the insulation and a 5 mm creep zone. MOPP requirements are even higher. In this case, a dielectric strength of the insulation at 1500 VAC and a 4 mm creep zone are defined for 1 H MOPP and a dielectric strength of the insulation at 4000 VAC and an 8 mm creep zone of are required for 2 H MOPP.

EN 60601-1 classifies network connections between medical devices and a communications network of a medical facility as a possible source of danger. For example, a leakage current occurring because of potential differences between ground connections of different electrically interconnected network components may constitute a threat. The leakage current can distort measurement results of devices for monitoring patients and thereby result in misdiagnoses and faulty treatments or may even cause malfunction of control devices of equipment for treating patients.

Potential differences may come about, for example, due to faulty electrical installation, moisture in the electrical installation or cables that were defective from the beginning or became defective.

Use of network cables without electrical shielding does not constitute a reliable solution to the problem because adequate galvanic insulation cannot be ensured in this way. Furthermore, such network cables have little or no poor imperviousness to incident interference and may even emit interference itself, which is absolutely unacceptable in medical applications.

Network insulators are one possibility for galvanically separating and isolating network connections in such a way that no fault currents flow and/or no foreign voltages can be applied. Network insulators for use in a local area network (LAN) are components connecting each pair of conductors of a network line arriving at an input of the network insulator to a corresponding pair of conductors at an output of the network insulator via a 1:1 signal transmitter. The signal transmitter here couples the input end and the output end magnetically like a transformer, wherein the transmitter ensures an adequate dielectric strength between the input end and the output end to fulfill the specifications of EN 60601-1. However, it would be desirable to provide an alternative approach for a communications interface of a medical device to protect patients and/or operating personnel from hazardous electrical voltages and/or leakage currents and that has a reduced sensitivity to electromagnetic interference.

### SUMMARY

In an exemplary embodiment, the invention provides a medical device. The medical device includes: a housing; electrical and/or electronic components arranged inside the housing; a user interface; and a first receiving device, accessible outside of the housing, of a first communications interface, wherein the first communications interface is connected to a communications network; wherein the electrical and/or electronic components of the medical device are communicatively connected to the first communications interface; wherein a communications link between the communications network and the electrical and/or electronic components of the medical device has at least two segments; and wherein at least one of the at least two segments is configured for transmission of communication signals in an electrically non-conductive manner and has an electrical separation between an input of the at least one segment and an output of the at least one segment with a predetermined first dielectric strength

In another exemplary embodiment, the invention provides a system, comprising: a medical device; an optical connection, configured to carry optical communication signals; and a converter device, configured to converter electrical signals into optical signals and/or optical signals into electrical signals; wherein the medical device comprises: a housing; electrical and/or electronic components arranged inside the housing; a user interface; and a first receiving device, accessible outside of the housing, of a first communications interface, wherein the first communications interface is connected to a communications network; wherein the electrical and/or electronic components of the medical device are communicatively connected to the first communications interface; wherein a communications link between the communications network and the electrical and/or electronic components of the medical device has at least two segments; and wherein at least one of the at least two segments is configured for transmission of communication signals in an electrically non-conductive manner and has an electrical separation between an input of the at least one segment and an output of the at least one segment with a predetermined first dielectric strength wherein the first receiving device is configured to receive a first end of the optical connection; wherein the converter device comprises a power supply at a second end of the optical connection, wherein the power supply is supplied with power from a power source situated outside of the medical device.

### BRIEF DESCRIPTION OF THE DRAWINGS

Exemplary embodiments of the present invention will be described in even greater detail below based on the exemplary figures. The invention is not limited to the exemplary embodiments. All features described and/or illustrated herein can be used alone or combined in different combinations in embodiments of the invention. Features and advantages of various embodiments of the present invention will become apparent by reading the following detailed description with reference to the attached drawings which illustrate the following:

FIG. 1 shows a medical device with a first known embodiment of a communications interface;

FIG. 2 shows a medical device with a second known embodiment of a communications interface;

FIG. 3 shows a first exemplary embodiment of a medical device with a communications interface configured up for protection of patients and operators;

FIG. 4 shows a first exemplary embodiment of a medical device with a communications interface configured up for protection of patients and operators;

FIG. 5 shows a third exemplary embodiment of a medical device with a communications interface configured up for protection of patients and operators;

FIG. 6 shows a first exemplary embodiment of a system with a medical device having a communications interface configured up for protection of patients and operators;

FIG. 7 shows a second exemplary embodiment of a system with a medical device having a communications interface configured up for protection of patients and operators; and

FIG. 8 shows a fourth exemplary embodiment of a system with a medical device having a communications interface configured up for protection of patients and operators.

### DETAILED DESCRIPTION

Exemplary embodiments of the invention provide a communications interface of a medical device configured for protecting patients and/or operating personnel from hazardous electrical voltages and/or leakage currents. Exemplary embodiments of the invention further provide a communications interface of a medical device having reduced sensitivity to electromagnetic interference.

Some embodiments of an arrangement for a communications interface described below with reference to a medical device provide for protection of patients and/or operating personnel from hazardous electrical voltages.

Some embodiments of an arrangement for a communications interface described below with reference to a medical device provide for reduced sensitivity to electromagnetic interference.

Some embodiments of an arrangement for a communications interface described below with reference to a medical device provide for protection of patients and/or operating personnel from hazardous electrical voltages and for reduced sensitivity to electromagnetic interference.

In the interests of better legibility and comprehensibility, the present disclosure will in some places describe only the conversion of an electrical signal into an optical signal or the conversion of an optical signal into an electrical signal. It is self-evident that such a conversion also takes place in the other direction in the case of a bidirectional communications interface.

In an exemplary embodiment, a medical device with a communications interface comprises at least one housing, electrical and/or electronic components arranged inside the housing, and a user interface. The user interface may comprise a display and operating elements, which are operated by touch, to operate the medical device. One example of such a medical device is a dialysis machine, such as that described in detail further above.

The medical device also has a first receiving device, which is accessible outside of the housing and is part of a first communications interface for receiving a proximal end of a medium that carries communication signals. The first receiving device, which is accessible from the outside, may be a bushing of a network connection, for example, and the medium carrying the communication signals is a corresponding network cable, for example, a cable for a local area network (LAN) according to the IEEE 802.3 standard (Ethernet) or an optical connecting line for a fiber-optic network, for example, according to any one of the standards of the IEC 61754 family

The first communications interface is communicatively connected to one or more processors, so that the one processor or the multiple processors can send and/or receive data. The first communications interface may also be implemented with the one processor or the multiple processors together as part of a system integrated on a computer chip (system-on-chip, SOC). One processor or multiple processors are also connected so they can communicate with volatile or nonvolatile memories, so that program instructions of computer programs as well as data can be read out, saved and processed.

The distal end of the medium carrying the communication signals is provided for connection to a corresponding second receiving device, which is connected via a communications network to a second communications interface. The second communications interface and/or the communications network are not part of the medical device here.

A communication link between the communications network and the electrical and/or electronic components of the medical device that are inside the device and are connected so they can communicate with the first communications interface has at least two segments. At least one of the segments transmits communication signals in an electrically non-conductive manner and has an electrical and/or galvanic separation between the input and output and/or between the output and input, respectively, of the segment with a predetermined first dielectric strength.

A communication connection can be established permanently or optionally via the communication link when the distal and proximal ends of the medium carrying the communication signals are connected to the respective receiving devices.

In one embodiment of the medical device, the segment transmitting communication signals in an electrically non-conductive manner is a fiber-optic data connection, for example. In the case of a fiber-optic data connection inside the device, electrical data signals arriving at the medical device are converted into optical data signals in a corresponding converter and sent to at least one processor or multiple processors or additional electrical and/or electronic components inside the medical device for further processing or forwarding, optionally after being converted back into electrical data signals via corresponding converters. The fiber-optic data connection may comprise monomode or multimode glass fibers or non-conductive multimode plastic fibers, also known as plastic optical fiber or POF. Step index fibers as well as gradient index fibers may also be used. A fiber-optic connection can also be established by the medium carrying the communication signals, and insulation of the touchable parts of the communications interface of the medical device is ensured by the fiber-optic connection and the first receiving device for receiving a medium carrying the optical communication signals. The first receiving device may be configured to receive fiber-optic connections according to one of the standards of the IEC 61754 family, for example.

The fiber-optic data connection may have a separate optical line for the transmitting and/or receiving directions for bidirectional operation in order to permit so-called full-duplex operation, in which a transmitter at one end of an optical line is connected to a receiver at the other end of the optical line. If the medium carrying the communication signals transmits optical signals, the first receiving device is configured to receive two light-conducting fibers accordingly. Full-duplex describes the option of transmitting and receiving at the same time. Alternatively, light of another wavelength sent over a shared optical line may be used for the transmitting direction and/or receiving direction (wavelength duplex and/or multiplex). Again in this case, a full-duplex mode is possible. Input and/or output of light of the respective wavelength take(s) place in corresponding combined transmitter-receiver modules (transceiver=transmitter-receiver). If full-duplex operation is not necessary, bidirectional data communication may also take place in so-called half-duplex operation, in which transmission and reception take place in alternation. To do so, data to be transmitted may be stored temporarily, until the respective transmitter can transmit the data. Alternatively, data to be transmitted can be created via synchronized control, so that storage of data at the transmitter end may be unnecessary. Control may be provided via a suitable protocol, which in this case may exclude simultaneous transmission.

In an exemplary embodiment of the medical device, the communication signals are transmitted optically in a spectrum including both infrared and/or visible light.

In an exemplary embodiment of the medical device, at least one segment of the communication zone, which transmits communication signals in an electrically non-conductive manner is disposed inside the medical device.

In an exemplary embodiment of the medical device, at least one segment of the communication link that transmits communication signals in an electrically non-conductive manner is disposed inside the medical device, and a circuit supplied with power from a first voltage supply is provided in or on the device for converting electrical signals into optical signals and/or optical signals into electrical signals. The first voltage supply is configured to ensure a galvanic and/or electrical separation of the circuit for conversion of electrical signals into optical signals and/or optical signals into electrical signals from other electrical and/or electronic components inside the device which have a dielectric strength corresponding at least to the predetermined first dielectric strength. The receiving device of the first communications interface, which is accessible from outside of the housing of the medical device, is configured to receive a medium carrying electrical communication signals. The converter circuit, which is separated electrically and/or galvanically from other electrical and/or electronic components inside the device, converts the electrical communication signals into optical communication signals and forwards them over a suitable line to another converter circuit inside the device. In the additional internal converter circuit, the optical signals are converted into electrical signals and sent at least to the one processor or the multiple processors or additional electrical or electronic components inside the medical device.

The first voltage power supply may in turn be supplied with power from a voltage power supply of the medical device or independently thereof by an external power source disposed outside of the medical device.

In the case of supplying power to the first voltage power supply from a voltage power supply of the medical device, the first voltage power supply may comprise a DC voltage converter (DC-DC converter) connected to a suitable voltage rail of a voltage power supply of the medical device.

In the case of supplying power to the first voltage power supply from an external power source, the external voltage power supply may comprise a corresponding network part connected to a power supply network provided at an installation site of the medical device.

In another exemplary embodiment, a voltage that is considered reliable is available at the installation site of the medical device, this voltage being lower than the line voltage, for example, a low DC voltage of 12 VDC of a nurse calling system, which is supplied to and/or looped through the medical device. In the latter case, a trigger element of the nurse calling system is connected to the medical device. If this low DC voltage is supplied at a sufficient power and/or at a low ohmage level, then the first voltage power supply can either be supplied directly to the circuit for converting electrical signals into optical signals and/or optical signals into electrical signals or supplied after a corresponding reduction or increase to a suitable or required voltage, for example, through a linear regulator or a switch converter. The voltage adjustment does not require any particular electrical and/or galvanic separation between the input and output and can therefore be implemented inexpensively.

Alternatively, the first voltage power supply can be supplied with electric power via the medium conducting the communication signals, for example, via “power over Ethernet” (PoE) together with data over an Ethernet line through the communications network. The power supply voltage can be fed into the Ethernet line at a central location in the communications network, for example, a router or switch set up for this purpose. In another exemplary embodiment, the feed of the power supply voltage into the Ethernet line may take place only in the vicinity of the installation site of the medical device, for example, directly at the network terminal of the communications network. The latter approach can subsequently be implemented easily and without any great effort.

In an exemplary emdodiment, a system comprises the medical device described above, wherein the first receiving device, which is accessible from outside of the housing of the metidal device is configured to receive a proximal end of an optical connecting line of a fiber-optic network, convert electrical data signals into optical data signals, and/or convert optical data signals into electrical data signals. The system receives electrical data signals from the second communications interface connected to the communications network at the device, which is connected to the distal end of the optical connecting line for converting electrical data signals into optical data signals and conducting them over the optical connecting line to the first accessible receiving device outside of the housing of the medical device. A converter is communicatively connected to the first receiving device of the medical device and converts the optical signals into electrical signals and supplies them for forwarding to the one processor or the multiple processors as well as optionally additional internal electrical and/or electronic components of the medical device. Similarly, the converter that is communicatively connected to the receiving device of the medical device receives electrical signals from the one processor or the multiple processors as well as optionally the additional internal electrical and/or electronic components of the medical device and converts them into optical signals. These optical signals are sent over the optical connecting line to the device connected to the distal end of the optical connecting line for converting electrical data signals into optical data signals and/or optical data signals into electrical data signals, where they are converted into electrical data signals and forwarded to the second communications interface connected to the communications network.

The device for converting electrical signals into optical signals and/or optical signals into electrical signals comprises a second power supply voltage, which is supplied with power from a power source situated outside of the medical device. The power source situated outside of the medical device may be, for example, a power supply unit that is connected to a power supply network available at the installation site. It is also possible to use a DC power supply voltage available at the installation site, for example, associated with a nurse calling system.

In an exemplary embodiment of the system, the power is supplied to the second power supply voltage over the second communications interface associated with the communications network, for example, via PoE over the medium conducting the communication signals, said medium being arranged between the second communications interface associated with the communications network and the device for converting electrical data signals into optical data signals and/or optical data signals into electrical data signals.

The PoE power feed, as in the embodiment described above, may take place at a central location in the communications network, for example, in a router or switch set up for this purpose. In another example, the power supply voltage may be fed into the Ethernet line only in the vicinity of the installation site of the medical device, for example, directly at the network connection of the communications network. The latter approach can also be implemented subsequently and easily without any great complexity, as already mentioned above.

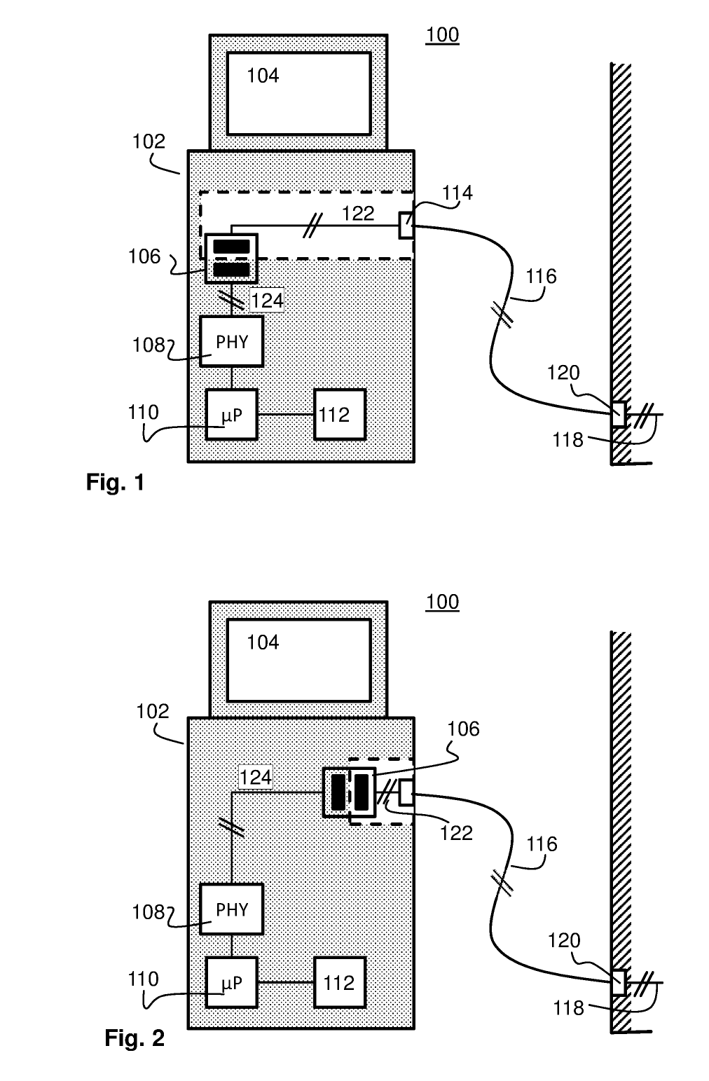

FIG. 1 shows a medical device 100 with a first known embodiment of a communications interface. The medical device 100 comprises a housing 102, in which electrical and/or electronic components are arranged. The electrical and/or electronic components are represented in the figure by a data transmission device 106, a circuit 108 (PHY), which implements a transfer point between a logic part of the communications interface and the physical transmission, a microprocessor 110 (μP) and a memory 112. The housing may also include additional electrical and/or electronic components, for example, pumps, actuators, control circuits and the like. The medical device 100 also has a user interface 104, represented by a display screen in the figure. The user interface may include additional elements, for example, switches, buttons, levers, regulators, etc. The medical device 100 also has a receiving device 114 for a network cable 116, for example, a LAN cable connecting the medical device 100 to a second communications interface, which does not belong to the medical device, via a communications network 118. The network cable 116 may be connected to the communications network 118 via a second receiving device 120, for example, a network junction box.

The network cable 116 establishes an electrically conductive connection between the medical device 100 and devices and equipment connected to the communications network 118. Data transmission device 106 creates a functional insulation of the network interface, for example, in order to keep DC cycle disruptions away from the first communications interface. Furthermore, the data transmission device 106 is situated far inside the medical device 100. Connecting line 122, connecting the receiving device 114 for the network cable 116 to the data transmission device 106 internally is electrically connected to the network cable 116 and carries the same voltage as the latter. If the insulation is damaged, then parts of the medical device 100 may become live (carrying voltage) and may constitute a threat to an operator or a patient connected to the medical device 100. The region at risk of DC voltage disruptions applied to the network cable 116 is indicated by the region outlined with a broken line and not colored in gray in the figure. Furthermore, electromagnetic interference received over the network cable 116 can be carried through the connection to the connecting line 122 into the interior of the housing 102 of the medical device 100, where it can have an adverse effect on measurement and/or control circuits, among other things.

FIG. 2 shows a medical device 100 with a second known embodiment of a communications interface. The medical device 100 shown in this figure corresponds largely to the medical device 100 described with respect to FIG. 1. In contrast with the medical device 100 described with reference to FIG. 1, the data transmission device 106 is placed very close to the receiving device 114 for the network cable 116, so that the connecting line 122 is very short and may optionally be omitted entirely. However, since the data transmission device 106 results in very little or no filtering effect with respect to the electromagnetic interference input into the network line 116, the interference can propagate over the connecting line 124 in the interior of the housing 102 of the medical device 100 and cause unwanted effects there. Furthermore, the functional insulation of the data transmission device 106 does not provide adequate protection of patients or operators against overvoltages, exactly as is the case with the embodiment illustrated in FIG. 1. As in FIG. 1, the region at risk of DC voltage disturbances applied to network cable 116 is indicated in the figure by the region that is bordered by the broken line and is not shaded gray.

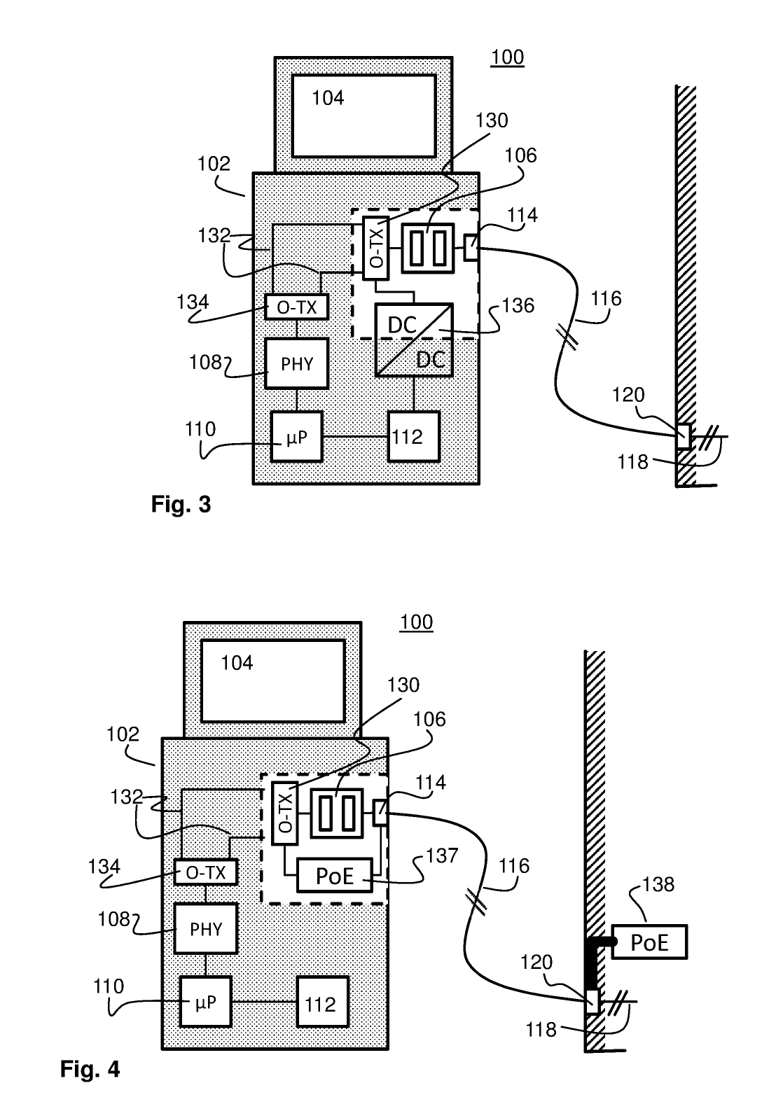

FIG. 3 shows a first embodiment of a medical device with a communications interface configured to protect patients and operators. The medical device 100 comprises a housing 102 which accommodates the electrical and/or electronic components. The electrical and/or electronic components are represented in the figure by a circuit 108 (PHY) which implements a transfer point between a logic part of the communications interface and the physical transmission, and also represents a microprocessor 110 (μP) and a memory 112. The housing 102 may further include additional electrical and/or electronic components, for example, pumps, actuators, control circuits and the like. The medical device 100 also has a user interface 104, represented in the figure by a display screen which may also be touch-sensitive, i.e., a touchscreen. The user interface may comprise additional elements, for example, switches, buttons, levers, regulators, etc. The medical device 100 also has a receiving device 114 for a network cable 116, for example, a LAN cable, connecting the medical device 100 via a communications network 118 to a second communications interface not associated with the medical device.

The network cable 116 establishes an electrically conductive connection between the receiving device 114 for the network cable 116 and devices and equipment connected to the communications network 118. The data transmission device 106 connected to the receiving device 114 produces a functional insulation of the network interface, for example, in order to keep DC cycle interference away from the first communications interface, and it may be omitted, depending on the type and arrangement of the other circuit parts. Electrical signals sent over the network cable 116 and the receiving device 114 to the medical device are converted from electrical signals into optical signals in a first circuit (O-TX) 130 for conversion of electrical signals into optical signals and are sent over a medium 132, conducting optical signals to a second circuit (O-TX) 134 for converting optical signals into electrical signals, where they are converted and relayed over additional interface components (PHY) 108 to the microprocessor 110. Accordingly, signals sent from the microprocessor 110 pass over the additional interface components 108 to the second circuit (O-TX) 134 for conversion of electrical signals into optical signals, then over the medium 132 that conducts optical signals to the first circuit (O-TX) 130 for conversion of optical signals into electrical signals, are converted there and sent via the receiving device 114 to the network cable 116. The medium 132 conducting optical signals may be a medium that is used jointly for both communication directions or may comprise two separate media, each of which is used for signals of one communication direction.

A power supply to the first circuit 130 is provided by way of a DC voltage converter (DC/DC) 136, which is supplied with power by an internal power supply unit and ensures the electrical and/or galvanic separation with the predetermined first dielectric strength. The DC voltage converter 136 may also supply one or more additional power supply voltages as needed. The first circuit 130, the DC voltage converter 136 and optionally the data transmission device 106 are preferably set up near one another in space and at a distance from other electrical and/or electronic components situated in the housing 102 or separated from them by other structural measures, for example, by non-conductive and/or shielding encapsulation, so that electrical and/or galvanic separation is ensured with the given first dielectric strength.

In the exemplary embodiment described above, dangerous voltages can travel over the communications network and/or electrical devices and equipment associated therewith only up to the input circuit, which is separated by an adequate distance or other structural measures, for example, a non-conductive and/or shielding encapsulation, and cannot have an effect on other internal components of the medical device. Propagation of interference input over the network cable 116 in the interior of the medical device is reduced or prevented. The area at risk due to the dangerous voltages and interference applied to the network cable 116 is indicated by the area shown with dashed lines but not shaded in gray.

FIG. 4 shows a second exemplary embodiment of a medical device with a communications interface configured for protections of patients and operators. This embodiment is similar in most elements to the embodiment described with reference to FIG. 3. In contrast to the embodiment described previously with reference to FIG. 3, in this embodiment, power is supplied to the first circuit 130 via an external power source, wherein the network cable 116 also conducts electricity in addition to the communication signals. An example of supplying power via a network cable is PoE (power over Ethernet). Accordingly, the DC voltage converter 136 from FIG. 3 is replaced in the present FIG. 4 by a circuit 137 for separating power and data, which may in turn include one or more DC converters.

The first circuit 130, the circuit 137 for separation of power and data and optionally the data transmission device 106 are preferably disposed in proximity to one another and at such a distance from other electrical and/or electronic components in the housing 102 or separated from them by other structural measures, for example, by non-conductive and/or shielding encapsulation, so that the electrical and/or galvanic separation is ensured with the predetermined first dielectric strength.

Supplying electricity to circuits over a network cable includes feeding electricity into the network cable at some location. Such circuits are available commercially for PoE and are also known as PoE injectors. The figure illustrates a PoE injector 138 connected to the second receiving device 120 and feeds electricity at this point into the network cable to supply power to the first circuit 130. Alternatively, a PoE injection may also take place in a central network component, for example, a suitably configured router or switch.

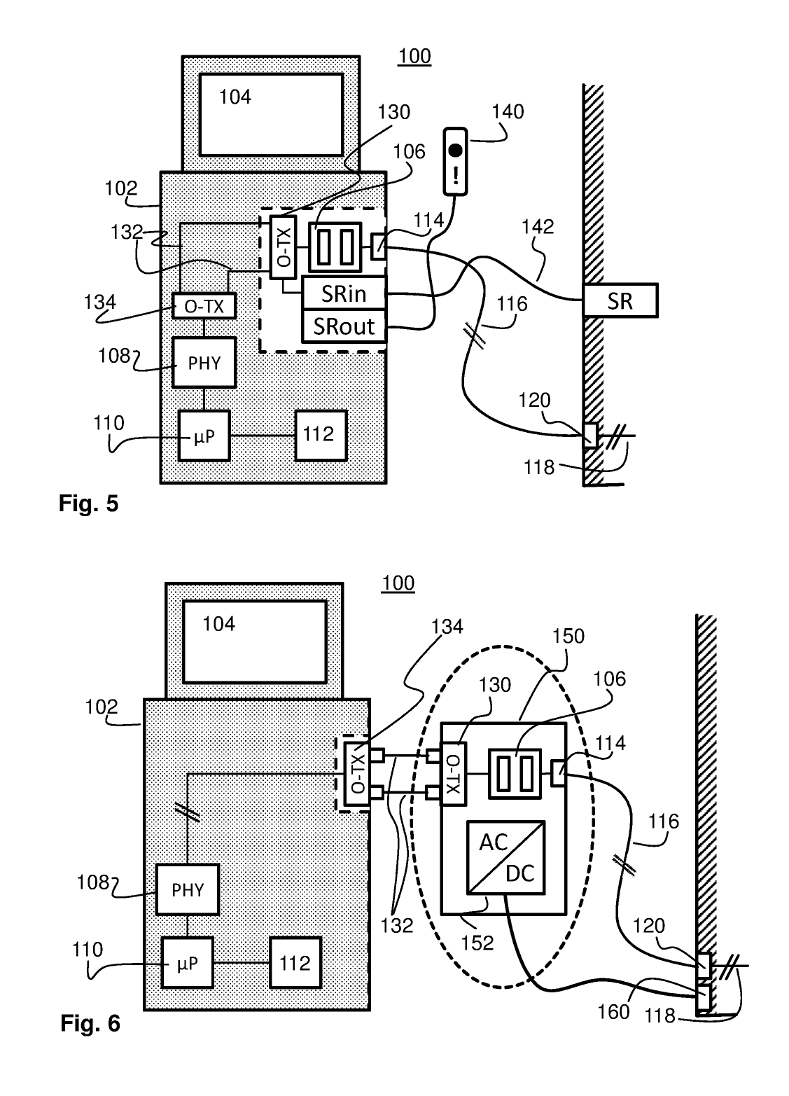

FIG. 5 shows a third exemplary embodiment of a medical device with a communications interface configured for protection of patients and operators. This embodiment is the same in most elements as the embodiment described with reference to FIG. 4. In contrast with the embodiment described previously with reference to FIG. 4, the power supply of electricity to the first circuit 130 from an external power source does not take place via the network cable 116, but instead in this embodiment, power is supplied via another external power source, which supplies a low electrical voltage that is classified as safe with a sufficient electrical isolation for protection of patients and operators, for example, a nurse calling system.

A release switch 140 for the nurse calling system is usually connected to a corresponding terminal SR at a treatment station via a plug connection. The release switch 140 may also include additional operating elements, for example, for operation of electrical facilities in a room, for example, for lighting or for control of blinds or the like (not shown in the figure). The room-side connection SR may be disposed in or on a wall of the room or on a so-called connection panel and/or a connection column, for example, the latter providing several connections for electricity, data connections, oxygen and/or compressed air supply and the like, which are provided via the treatment station. FIG. 5 shows the release switch 140 connected to a corresponding connection SRout, which is disposed in or on the medical device 100. The medical device 100 has a connection SRin connected to the room-side connection SR of the nurse calling system by a connecting line 142. The nurse calling system provides a power supply voltage for the first circuit 130, which is disconnected in the medical device 100. The connections for the release switch 140 are looped through from the connection SRin to the connection SRout.

The first circuit 130, connections SRin, SRout and optionally the data transmission device 106 are preferably disposed in spatial proximity to one another and at a distance from other electrical and/or electronic components in the housing 102 or separated from them by other structural measures, for example, by non-conductive and/or shielding encapsulation, so that the electrical and/or galvanic separation with the predefined first dielectric strength is ensured.

FIG. 6 shows a first exemplary embodiment of a system with a medical device with a communications interface configured for the protection of patients and operators. In this embodiment of the system, the first receiving device, which is accessible outside of the housing 102 of the medical device 100 is for receiving a proximal end of an optical signal-conducting medium 132. The first receiving device may comprise a suitably configured second circuit (O-TX) 134 for converting optical signals into electrical signals, converting the incoming optical signals and forwarding them to the microprocessor 110 over internal lines and interface components (PHY) 108. For communication in the opposite direction, the second circuit 134 converts electrical signals coming from the microprocessor 110 and arriving at the second circuit 134 over internal lines as well as interface components (PHY) 108 into optical signals and makes them available outside of the medical device 100.

The system in this embodiment comprises a device 150 situated outside of the medical device 100 and having a first circuit 130 for conversion of electrical signals into optical signals and/or for conversion of optical signals into electrical signals which is connected via a medium 132 that conducts optical signals to the second circuit (O-TX) 134 for conversion of optional signals into electrical signals and/or for conversion of electrical signals into optical signals. The medium 132 conducting the optical signals may be a medium used jointly for both communication directions or may comprise two separate media, each of which is used for signals of one communication direction.

The first circuit 130 converts optical signals coming from the medical device 100 into electrical signals and forwards them to the communications network 118. For this purpose the first circuit 130 may be connected to the communications network 118 via a receiving device 114 for a network cable 116 and the network cable 116 to the communications network 118. Network cable 116 establishes an electrically conducting connection between the receiving device 114 for the network cable 116 and devices and equipment connected to the communications network 118. The data transmission device 106 connected to the receiving device 114 causes functional isolation of the network interface, for example, to keep DC interference away from the first communications interface and may be omitted, depending on the type and arrangement of the additional circuit parts.

The first circuit 130 and circuit parts electrically connected thereto are part of a device 150 arranged in a housing. The housing provides protection against contact with voltage-carrying parts. A power supply of the first circuit 130 is provided via a network part 152 which is independent of the medical device 100 and which ensures an electrical and/or galvanic separation with the predefined first dielectric strength. The network part 152 may be arranged in the housing or may be embodied as an external plug network part. The network part 152 is supplied with electricity from a network voltage connection 160 which is supplied locally. In an alternate exemplary embodiment, power is supplied to the first circuit 130 and the additional circuit parts via the network cable 116, for example, via PoE, as described above with reference to FIG. 4.

The first circuit and the components connected directly to it, which are indicated by the dashed line in the figure, can be arranged close to the medical device 100 or close to the room-side network connections and network voltage connections, depending on the respective length of the network cable 116 and the medium 132 conducting the optical signals.

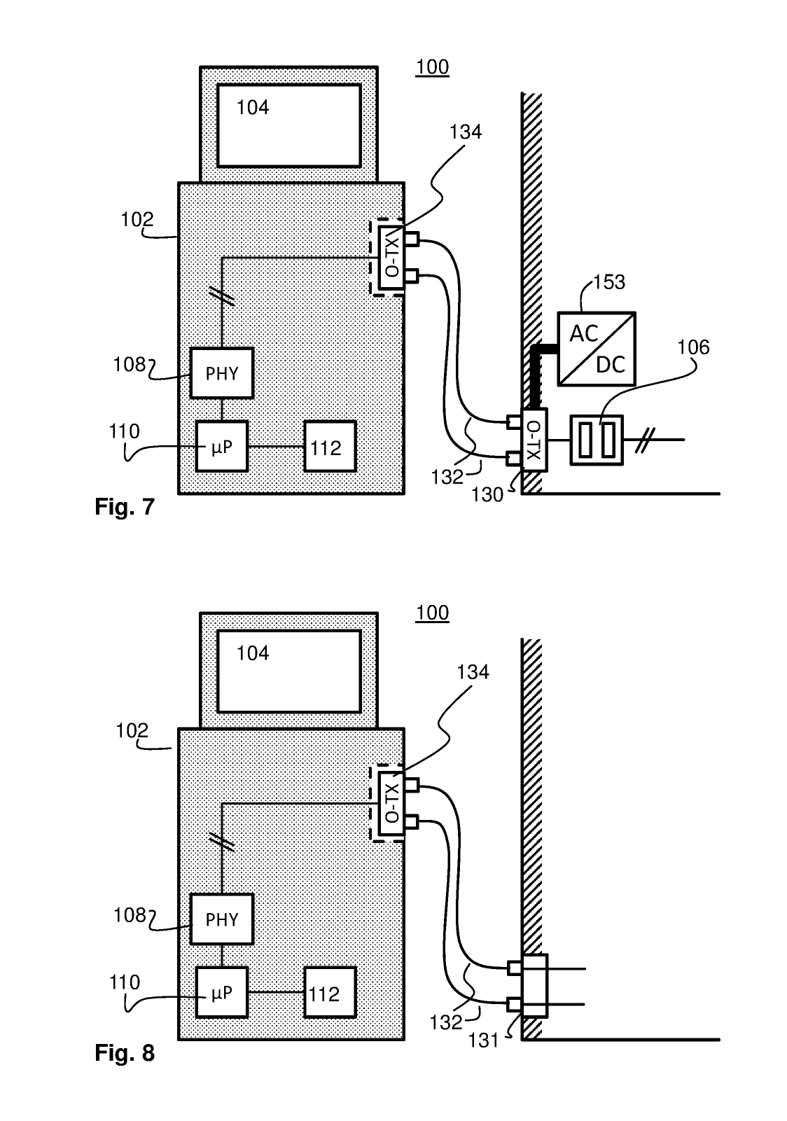

FIG. 7 shows a second embodiment of a system with a medical device with a communications interface configured for protecting patients and operators. The system corresponds largely to the system described with respect to FIG. 6. In contrast with that, the first circuit 130 and the circuit parts connected electrically thereto are not arranged in a separate housing but instead are a fixed part of a room-side installation. Power is supplied to the first circuit 130 via a network part 153 provided in the installation and ensures an electrical and/or galvanic separation with the predetermined first dielectric strength on the installation end.

FIG. 8 shows a fourth exemplary embodiment of a medical device 100 with a communications interface configured for protection of patients and operators. The medical device 100 corresponds to the medical device described with respect to FIG. 6. In this embodiment, a connection 131 is provided on an optical communications network. A medium 132 that carries optical signals connects the second circuit (O-TX) 134 of the medical device 100 to the room-side connection 131.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A medical device, comprising:
a housing;
electrical and/or electronic components arranged inside the housing;
a user interface; and
a first receiving device, accessible outside of the housing, of a first communications interface, wherein the first communications interface is connected to a communications network;
wherein the electrical and/or electronic components of the medical device are communicatively connected to the first communications interface;
wherein a communications link between the communications network and the electrical and/or electronic components of the medical device has at least two segments; and
wherein at least one of the at least two segments is configured for transmission of communication signals in an electrically non-conductive manner and has an electrical separation between an input of the at least one segment and an output of the at least one segment with a predetermined first dielectric strength.

2. The medical device according to claim 1, wherein the at least one segment of the communications link is configured tor optical transmission of communication signals.

3. The medical device according to claim 1, wherein the at least one segment of the communications link is configured tor optical transmission of communication signals in a spectrum comprising infrared light and/or visible light.

4. The medical device according to claim 1, wherein the at least one segment of the communications link is disposed inside the housing of the medical device.

5. The medical device according to claim 4, further comprising:
a media converter, disposed in or on the medical device and supplied with power from a power supply, wherein the first media converter is configured to convert electrical signals into optical signals and/or optical signals into electrical signals;
wherein the power supply is configured to provide electrical isolation of the media converter from electrical and/or electronic components outside of the medical device, with a dielectric strength corresponding at least to the predetermined first dielectric strength.

6. The medical device according to claim 4, further comprising:
a media converter, disposed in or on the medical device and supplied with power from a power supply disposed outside of the medical device, wherein the supplied power is provided at a voltage level below a predetermined maximum voltage level.

7. The medical device according to claim 4, further comprising:
a media converter, disposed in or on the medical device, configured to convert electrical signals into optical signals and/or optical signals into electrical signals, wherein the media converter is supplied with power via a connection that also carries communication signals, and wherein the supplied power is provided at a voltage level below a predetermined maximum voltage level.

8. The medical device according to claim 1, wherein the first receiving device is configured to receive a connection carrying optical communication signals.

9. A system, comprising:
a medical device, wherein the medical device comprises:
a housing;
electrical and/or electronic components arranged inside the housing;
a user interface; and
a first receiving device, accessible outside of the housing, of a first communications interface, wherein the first communications interface is connected to a communications network;
an optical connection, configured to carry optical communication signals; and
a converter device, configured to converter electrical signals into optical signals and/or optical signals into electrical signals;
wherein the electrical and/or electronic components of the medical device are communicatively connected to the first communications interface;
wherein a communications link between the communications network and the electrical and/or electronic components of the medical device has at least two segments; and
wherein at least one of the at least two segments is configured for transmission of communication signals in an electrically non-conductive manner and has an electrical separation between an input of the at least one segment and an output of the at least one segment with a predetermined first dielectric strength;
wherein the first receiving device is configured to receive a first end of the optical connection;
wherein the converter device comprises a power supply at a second end of the optical connection, wherein the power supply is supplied with power from a power source situated outside of the medical device.

10. The system according to claim 9, wherein the power supply is supplied with power via a connection that also carries communication signals between a second communications interface, corresponding to the communications network, and the converter device.
